(19) United States
(12) Patent Application Publication
(10) Pub. No.: US 2023/0012767 A1
(43) Pub. Date: Jan. 19, 2023

(54) TORQUE-LIMITING DEVICE FOR MANUAL INSERTION OF SYRINGE PLUNGER RODS AND METHODS OF USING SAME

(71) Applicant: Merck Sharp & Dohme LLC, Rahway, NJ (US)

(72) Inventors: Wail Rasheed, Union, NJ (US); Raymond K. Seto, Warren, NJ (US); Ophelia Wells, Easton, PA (US); Cassie Megna, New Milford, NJ (US); Eduardo Cristofolli, Elizabeth, NJ (US)

(73) Assignee: Merck Sharp & Dohme LLC, Rahway, NJ (US)

(21) Appl. No.: 17/782,946

(22) PCT Filed: Dec. 11, 2020
(86) PCT No.: PCT/US2020/064431

§ 371 Date: Jun. 6, 2022

Related U.S. Application Data
(60) Provisional application No. 62/948,503, filed on Dec. 16, 2019.

(51) Int. Cl. A61M 5/315; A61M 5/31; A61M 5/20

(52) U.S. Cl. CPC A61M 5/31515; A61M 5/3129; A61M 5/2033; A61M 2207/10

(57) ABSTRACT

A torque-limiting device comprises a plunger rod holder defining a piston cavity for receiving at least a portion of a plunger rod, a moveable stop element, a dowel disposed adjacent the stop element, a spring disposed adjacent the dowel, and a syringe barrel holder defining a barrel cavity for receiving at least a portion of a syringe barrel.

### FIELD OF THE INVENTION

The present disclosure relates generally to assembling syringes. More specifically, the present disclosure relates to torque-limiting devices and related methods that allow for easier and safer assembly of syringe components.

### BACKGROUND OF THE INVENTION

Manufacturing pre-filled syringes in small volumes (e.g., for clinical trials) often requires manual insertion of plunger rods into a syringe barrel due to a lack of small-scale automated solutions. Manually inserting and twisting a plunger rod into the threads of a plunger stopper can potentially result in over-torquing the plunger rod. Over-torquing the plunger rod may, in turn, displace the plunger stopper, putting the safety and quality of the product or medicament within the syringe at risk. Alternatively, under-torquing the plunger rod may result in accidental detachment.

Thus, there exists a need for devices that improve upon and advance the methods of assembling various components of syringes, such as pre-filled syringes.

### SUMMARY OF THE INVENTION

In one embodiment, a torque-limiting device comprises a plunger rod holder defining a piston cavity for receiving at least a portion of a plunger rod, a moveable stop element, a dowel disposed adjacent the stop element, a spring disposed adjacent the dowel, and a syringe barrel holder defining a barrel cavity for receiving at least a portion of a syringe barrel.

In another embodiment, the spring of the torque-limiting device described above is mounted about the dowel and the dowel is configured to apply a force onto the stop element.

In another embodiment, the stop element of the torque-limiting device described above is configured to move closer to, and away from, the plunger rod.

In another embodiment, the plunger rod holder and the syringe barrel holder of the torque-limiting device described above are unitarily formed.

In another embodiment, the plunger rod holder and the syringe barrel holder of the torque-limiting device described above are coupled together.

In another embodiment, the stop element of the torque-limiting device described above is configured to move along a longitudinal axis of the spring.

In another embodiment, the piston cavity of the torque-limiting device described above has a first height, the barrel cavity has a second height, the first height is variable and the second height is fixed.

In another embodiment, the piston cavity of the torque-limiting device described above has a first height, the barrel cavity has a second height, both the first height and the second height are variable.

In another embodiment, the piston cavity of the torque-limiting device described above includes a flared end at a proximal-most end of the plunger rod holder.

In yet another embodiment, the piston cavity and the barrel cavity of the torque-limiting device described above are aligned with one another.

In one embodiment, a torque-limiting device comprises a holder having a piston cavity for receiving at least a portion of a plunger rod, and a barrel cavity for receiving at least a portion of a syringe barrel, a dowel moveable with respect to the holder, a spring mounted on the dowel, and a stop element disposed adjacent the dowel.

In another embodiment, the piston cavity and the barrel cavity of the torque-limiting device described above are aligned with one another.

In another embodiment, the spring of the torque-limiting device described above is configured to apply a force onto the stop element.

In another embodiment, the stop element of the torque-limiting device described above is configured to apply a force onto the plunger rod.

In another embodiment, the dowel of the torque-limiting device described above is at least partially housed within the holder and is translatable relative thereto.

In one embodiment, a syringe assembling device includes a first holder having a piston cavity for receiving at least a portion of a plunger rod and a retaining element moveable relative to first holder and configured to apply a force on an object disposed within the piston cavity.

In another embodiment, the syringe assembling device described above further comprises a second holder having a barrel cavity for receiving at least a portion of a syringe barrel.

In another embodiment, the first holder and the second holder of the syringe assembling device described above are unitary.

In another embodiment, the retaining element of the syringe assembling device described above comprises a translating dowel, the translating dowel being biased against the object disposed within the piston cavity.

In another embodiment, the retaining element of the syringe assembling device described above comprises a pivoting stop element, the translating dowel being configured and arranged to apply a force onto the stop element to move the stop element.

In another embodiment, the translating dowel and the stop element of the syringe assembling device described above are configured and arranged to apply a pre-determined torque onto the plunger rod disposed within the piston cavity.

### BRIEF DESCRIPTION OF THE DRAWINGS

Various embodiments of the presently disclosed torque-limiting devices are disclosed herein with reference to the drawings, wherein:

FIG. 1 is a schematic illustration of a pre-filled syringe;

FIGS. 2A-B are schematic side views of a torque-limiting device according to one embodiment of the disclosure, the device having the spring compressed to varying degrees;

FIG. 2C is a schematic illustration showing a stop element pressing on a piston;

FIGS. 3A-B are schematic front end, and side views of the torque-limiting device of FIG. 2A with a pre-filled syringe disposed in the cavities;

FIG. 4 is a schematic illustration of an assembled pre-filled syringe;

FIG. 5 is a photograph of a torque-limiting device being used to assemble a syringe; and

FIG. 6 is a schematic illustration of a torque-limiting device having modular features.

Various embodiments will now be described with reference to the appended drawings. It is to be appreciated that these drawings depict only some embodiments of the disclosure and are therefore not to be considered limiting of its scope.

### DETAILED DESCRIPTION OF THE INVENTION

Despite the various improvements that have been made to pre-filled syringes, conventional methods suffer from some shortcomings as discussed above.

There, therefore, is a need for further improvements to the devices and methods used to help facilitate proper and quicker assembly of syringe components. Among other advantages, the present disclosure may address one or more of these needs.

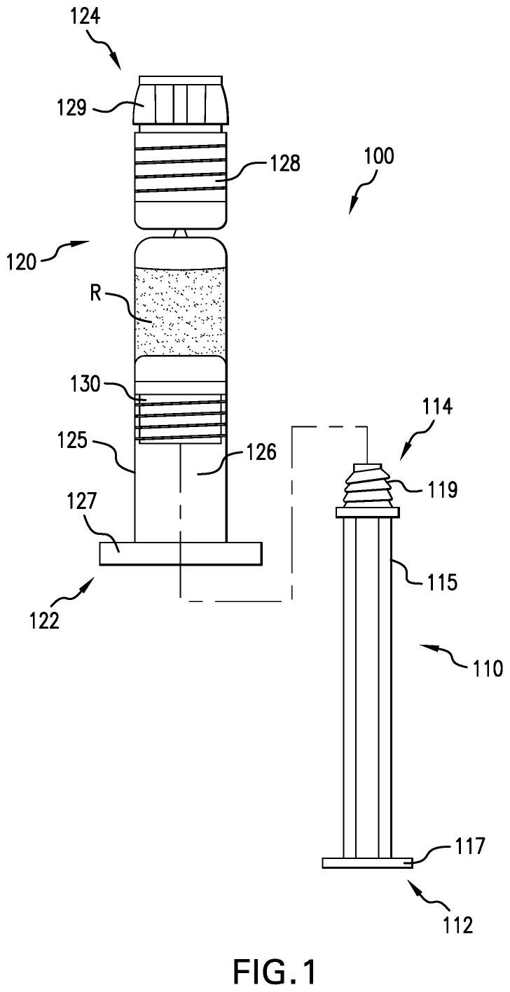

Reference is now made to FIG. 1, which shows a schematic illustration of a prefilled-syringe 100. Pre-filled syringe 100 generally comprises two main portions, a plunger rod 110 and a barrel 120. In one embodiment, barrel 120 contains a luer lock. In another embodiment, barrel 120 contains a stacked needle. Plunger rod 110 generally extends between a proximal end 112 and a distal end 114, and comprises an elongated piston 115 extending between a thumb press 117 and a coupler 119. In one embodiment, piston 115 has a cruciform cross-sectional shape. In one embodiment, thumb press 117 has a circular shape.

A cylindrical barrel 120 extends between proximal end 122 and distal end 124 and comprises a body 125 defining a lumen 126 for accepting a portion of plunger rod 110. As used herein, a “proximal” end means the end toward a user and a “distal” end means the end away from the user. Body 125 further comprises a flange 127 adjacent proximal end 122, and a removable cap 129 mateable with a hub 128 adjacent distal end 124. Body 125 defines a reservoir “R” that holds a medicament, drug, saline, or other substance. An internally threaded stopper 130 is disposed inside lumen 126 of body 125. In one embodiment, stopper 130 is made of an elastomeric material such as natural rubber, synthetic rubber, thermoplastic elastomers, or combinations thereof, and includes an opening to receive and mate with coupler 119 of plunger rod 110 by advancing the plunger rod inside the barrel lumen 126 and rotating at least one of coupler 119 and stopper 130 relative to the other. As discussed, problems may occur if the plunger rod is over-torqued, which may displace a portion of the syringe assembly (e.g., the stopper). Additionally, over-torquing the plunger rod may also damage the seal of the stopper exposing the contents of the syringe and compromise the sterility of the assembly and the medicament.

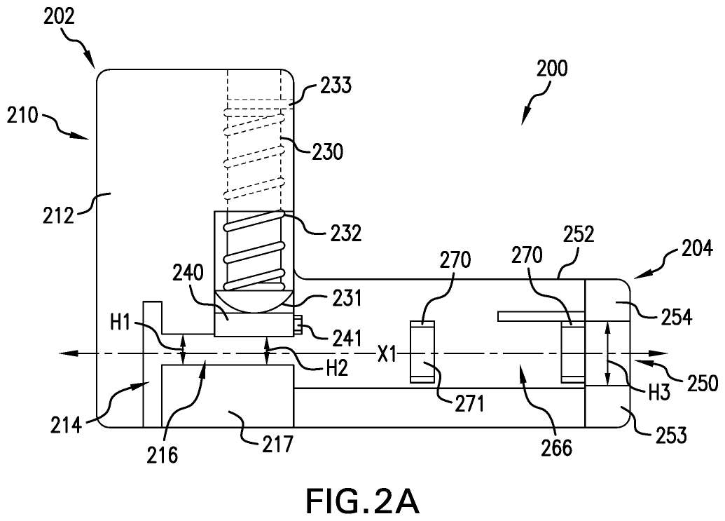

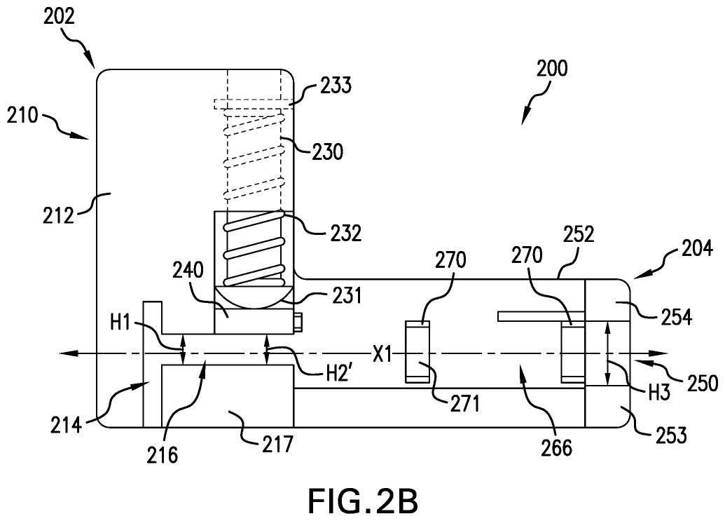

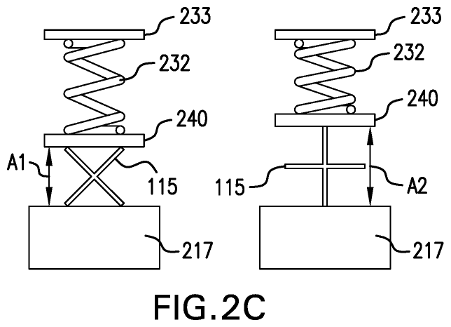

FIG. 2A illustrates one embodiment of a torque-limiting device 200 that extends between a proximal end 202 and a distal end 204. As shown, torque-limiting device 200 generally comprises a plunger rod holder 210 and a barrel holder 250. In the embodiment shown, plunger rod holder 210 and barrel holder 250 are unitarily formed, although it will be understood that the two components may be separately formed. Additionally, in one embodiment, the torque-limiting device comprises only the plunger rod holder 210 and no barrel holder.

Plunger rod holder 210 comprises a body 212 configured to hold a plunger rod, and the body may be formed of a metal, a plastic, wood, or any suitable rigid material. Body 212 may comprise a piston cavity 216 sized to accept a portion of a piston of a plunger rod, the piston cavity 216 being defined between a base 217 and a stop element 240, which is fixed to body 212 via pin 241. In one embodiment, stop element 240 comprises a flat stop plate. In another embodiment, stop element 240 comprises a curved or spherical (e.g., a ball) member. In one embodiment, stop element 240 may be capable of pivoting about pin 241 so that stop element 240 may rotate closer to, and away from, base 217. In one embodiment, stop element 240 may be translatable relative to base 217. Body 212 may further define a press void 214 that is configured to accept a thumb press of a plunger, flared press void and piston cavity 216 being in communication with one another. In one embodiment, base 217 may be fixed. Alternatively, base 217 may be adjustable or translatable up or down to vary the height H1 of piston cavity 216 to accommodate plungers of various diameters.

Disposed above piston cavity 216 is a translating dowel 230, which may be, at least partially, disposed inside body 212 and configured to move relative to the body. Dowel 230 may terminate in an enlarged head 231 configured and arranged to apply a force to the stop element. A spring 232 may be disposed about dowel 230 and be seated at head 231. Spring 232 may be fixed at one end (e.g., by pressing against backstop 233) so that it exerts a predetermined downward force on head 231, urging head 231 down toward piston cavity 216. It will be understood that other biasing elements may be used instead of spring 232, the biasing element being capable of exerting a predetermined downward force on stop element 240. A height H2 may be defined between support 217 and stop element 240. This height may change as the stop element 240 moves up or down as a result of the compression or expansion of spring 232. FIG. 2B shows the torque-limiting device 200 with spring 232 being slightly more compressed and the stop element 240 being spaced farther from base 217, resulting in a height H2′ that is larger than height H2.

FIG. 2C illustrates how the compression of the spring 232 relates to the cross-section of piston 115. As previously-noted, piston 115 may have a cruciform shape, and due to this shape, rotating the piston 115 may change the vertical component of the piston from A1 to A2, pushing up stop element 240 and compressing spring 232. Spring 232 may have a predetermined spring constant k1 and spring length that are chosen based on the size and/or shape of piston 115, or on the torque specified to properly mate the two components. Additionally, spring constant k1 and the height of the spring may also be chosen, at least partly based on the materials used for the plunger rod 110 and stopper 130.

Turning back to FIG. 2A, barrel holder 250 comprises a body 252 having a lower support 253, an upper support 254 and a barrel cavity 266 formed between lower and upper supports 253,254. In the embodiment shown, body 252 and body 212 are integrally formed as one. Barrel cavity may have a height H3 that is large enough to accept a portion of barrel 120 of a syringe. In one embodiment, height H3 is variable by making either lower support 253, or upper support 254, or both movable with respect to the other to accommodate barrels of different sizes. As shown, piston cavity 216 and barrel cavity 266 may be directly aligned with one another along transverse axis X1, and in communication with one another.

Optionally, one or more central bolsters 270 may be formed on body 212 or body 252 (or both) between the piston cavity 216 and the barrel cavity 266, each of the central bolsters 270 having an indented face 271 to accept at least a portion of the piston and/or the barrel and allow it to rest thereupon. In one embodiment, the bolsters 270 may rest on tracks and may be moveable along transverse axis X1.

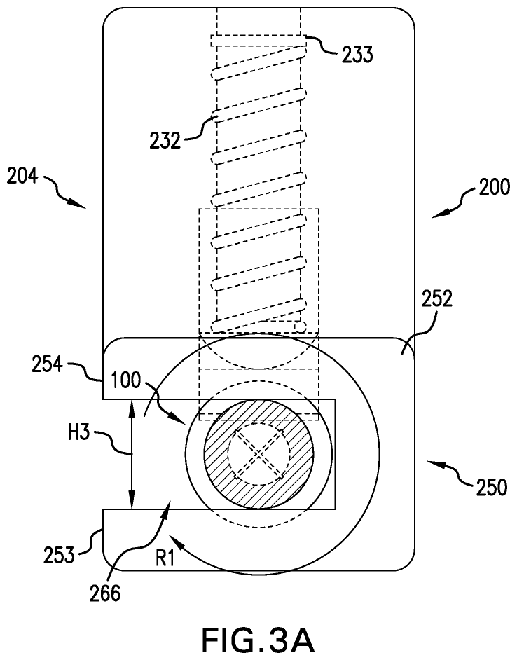

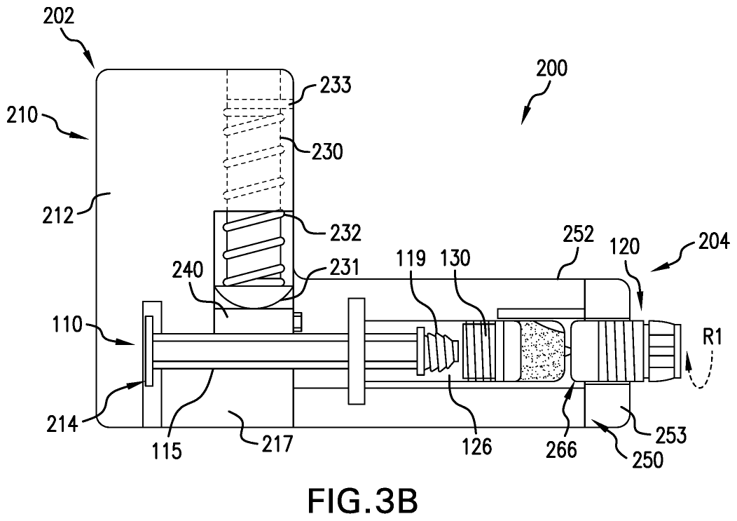

In use, torque-limiting device 200 may assist in coupling components of a pre-filled syringe together while ensuring that the problems associated with over-torquing are reduced. FIGS. 3A-B show a front-end view, and a side view of the torque-limiting device 200 being used with a pre-filled syringe 100. First, piston rod 115 of plunger 110 may be disposed in piston cavity 216 and barrel 120 may be disposed in barrel cavity 266, the two components being aligned with one another. Barrel 120 may be manually advanced toward piston 115 with coupler 119 entering lumen 126 and being urged toward stopper 130. With plunger rod 110 securely in place within piston cavity 216, spring 232 may push down on stop element 240 and urge it toward base 217. Rotating barrel 120 clockwise in the direction of arrow R1 may serve to join stopper 130 and coupler 119 as the barrel rotates, and the stop element 240 prevents the cruciform piston 115 from rotating. A user may continue to manually rotate the barrel 120 and continue to thread stopper 130 onto the static coupler 119. At a certain point, the release point, the barrel and the plunger will become sufficiently coupled together and begin to rotate simultaneously. At this release point, rotation of the barrel and the plunger will push up against stop element 240, and this force of the plunger will overcome the downward force of the spring, causing the plunger to rotate freely within piston cavity 216. Past this point, the stop element will no longer be able to retain or impede the rotating piston and further rotation of the barrel will cause the plunger to rotate within the piston cavity.

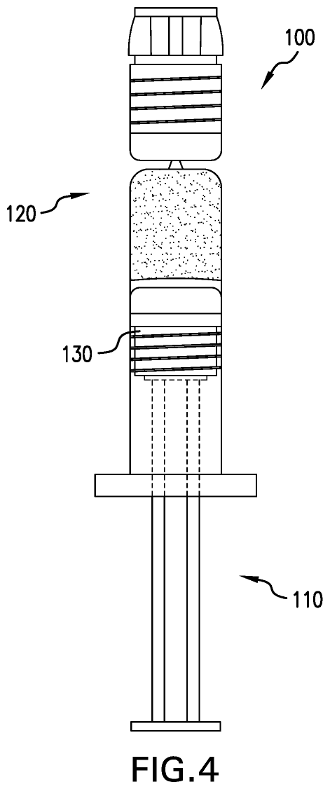

The characteristics of the spring (e.g., the spring constant k1) may be chosen to correlate to a specific torque to be applied from the barrel onto the plunger coupler. Because the torque required to properly mate the plunger and the stopper will at least partly depend on the materials of these components, the coatings disposed on the components, and/or the particular coefficients of friction associated with those materials and/or coatings, it will be understood that the spring constant of the spring may depend partly on the materials and/or coatings chosen. In one embodiment, the spring is chosen so that the downward force of the stop element is overcome when a torque is applied by the barrel onto the plunger rod. By using the torque-limiting device, the plunger and the barrel may be properly assembled (FIG. 4) without the drawbacks and risks described above.

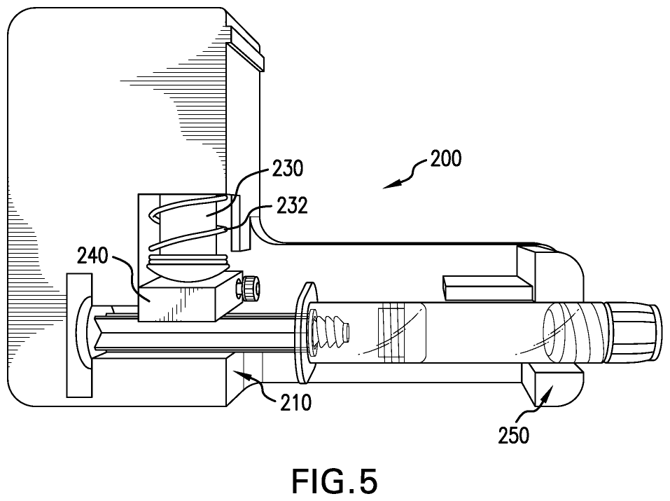

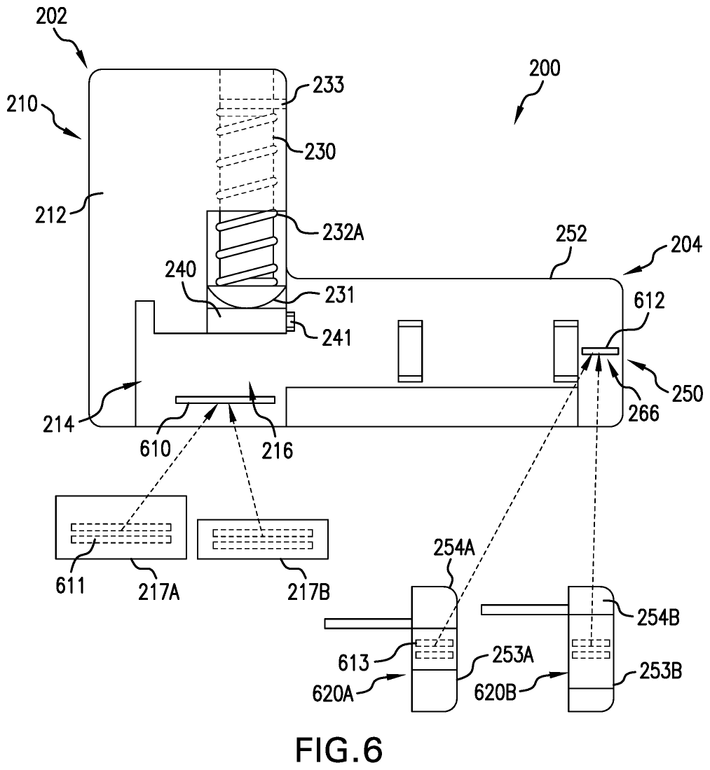

One embodiment of a torque-limiting device is shown in FIG. 5, where a single material is used to form an integral plunger rod holder and barrel holder. In the example shown, the body is formed of nylon, although it will be understood that other suitable rigid materials such as plastics and metals are also contemplated. In one embodiment, the body may be made by means of machining, casting, molding or additive manufacturing such as 3D printing. Additionally, certain features of the torque-limiting device may be modular, and one such embodiment is shown in FIG. 6. In this embodiment, the torque-limiting device 600 is similar to torque-limiting device of FIGS. 2-3, with certain modifications. For example, spring 232A is disposed about the dowel, but it may be replaceable with another spring with a higher or lower coefficient of friction. Additionally, certain elements may be removable or interchangeable to accommodate syringes of various sizes. In one embodiment, torque-limiting device 600 comprises a first projection 610, and various bases 217A,217B may have complementary clips 611 that are friction fit with the first projection 610. As will be appreciated, bases 217A,217B have different heights so that a user is able to choose the appropriately sized/shaped base to accommodate plungers of different sizes by changing the shape and/or size of the piston cavity 216. In another embodiment, the barrel holder comprises a second projection 612, and modular barrel jaws 620A,620B may have complementary clips 613, the modular barrel jaws having upper supports 254A,254B and lower supports 253A,253B of different sizes to accommodate barrels of different diameters by changing the shape and/or size of the barrel cavity 266. Other features of the torque-limiting device may also be modular or interchangeable, including, for example, the bolsters, stop element, dowel, and other components.

It is to be understood that the embodiments described herein are merely illustrative of the principles and applications of the present disclosure. For example, although the torque-limiting device has been described as being useful for manual assembly of syringe components, it will be understood that certain parts of the process may be automated. For example, the loading; of one or more of the syringe components (e.g., plunger, or barrel) may be automated. Additionally, the barrel assembly may be rotated using an automated process. Moreover, certain components (e.g., the barrel holder or central bolster) are optional, and the disclosure contemplates various configurations and combinations of the elements disclosed herein. It is therefore to be understood that numerous modifications may be made to the illustrative embodiments and that other arrangements may be devised without departing from the spirit and scope of the present disclosure as defined by the appended claims.

It will be appreciated that the various dependent claims and the features set forth therein can be combined in different ways than presented in the initial claims. It will also be appreciated that the features described in connection with individual embodiments may be shared with others of the described embodiments.

1. A torque-limiting device, comprising:
a plunger rod holder defining a piston cavity for receiving at least a portion of a plunger rod;
a moveable stop element;
a dowel disposed adjacent the stop element;
a spring disposed adjacent the dowel; and
a syringe barrel holder defining a barrel cavity for receiving at least a portion of a syringe barrel.

2. The torque-limiting device of claim 1, wherein the spring is mounted about the dowel and the dowel is configured to apply a force onto the stop element.

3. The torque-limiting device of claim 1, wherein the stop element is configured to move closer to, and away from, the plunger rod.

4. The torque-limiting device of claim 1, wherein the plunger rod holder and the syringe barrel holder are unitarily formed.

5. The torque-limiting device of claim 1, wherein the plunger rod holder and the syringe barrel holder are coupled together.

6. The torque-limiting device of claim 1, wherein the stop element is configured to move along a longitudinal axis of the spring.

7. The torque-limiting device of claim 1, wherein the piston cavity has a first height, and the barrel cavity has a second height, the first height being variable and the second height being fixed.

8. The torque-limiting device of claim 1, wherein the piston cavity has a first height, and the barrel cavity has a second height, both the first height and the second height being variable.

9. The torque-limiting device of claim 1, wherein the piston cavity includes a flared end at a proximal-most end of the plunger rod holder.

10. The torque-limiting device of claim 1, wherein the piston cavity and the barrel cavity are aligned with one another.

11. A torque-limiting device, comprising:
a holder having a piston cavity for receiving at least a portion of a plunger rod, and a barrel cavity for receiving at least a portion of a syringe barrel;
a dowel moveable with respect to the holder;
a spring mounted on the dowel; and
a stop element disposed adjacent the dowel.

12. The torque-limiting device of claim 11, wherein the piston cavity and the barrel cavity are aligned with one another.

13. The torque-limiting device of claim 11, wherein the spring is configured to apply a force onto the stop element.

14. The torque-limiting device of claim 13, wherein the stop element is configured to apply a force onto the plunger rod.

15. The torque-limiting device of claim 11, wherein the dowel is at least partially housed within the holder and is translatable relative thereto.

16. A syringe assembling device, comprising:
a first holder having a piston cavity for receiving at least a portion of a plunger rod; and
a retaining element moveable relative to the first holder and configured to apply a force on an object disposed within the piston cavity.

17. The syringe assembling device of claim 16, further comprising a second holder having a barrel cavity for receiving at least a portion of a syringe barrel, wherein the first holder and the second holder are unitary.

18. The syringe assembling device of claim 16, wherein the retaining element comprises a translating dowel, the translating dowel being biased against the object disposed within the piston cavity.

19. The syringe assembling device of claim 18, wherein the retaining element includes a pivoting stop element, the translating dowel being configured and arranged to apply a force onto the stop element to move the stop element.

20. The syringe assembling device of claim 19, wherein the translating dowel and the stop element are configured and arranged to apply a pre-determined torque onto the plunger rod disposed within the piston cavity.

21-22. (canceled)
